(19) United States
(12) Patent Application Publication
(10) Pub. No.: US 2016/0345933 A1
(43) Pub. Date: Dec. 1, 2016

(54) ULTRASOUND SYSTEM AND METHOD

(71) Applicant: Signostics Limited, Clovelly Park (AU)

(72) Inventors: Stewart BARTLETT, Goodwood (AU); Matthew McCARTHY, Dover Gardens (AU); Essa EL-AKLOUK, Hillcrest (AU); Andrew NIEMIEC, Seaton (AU)

(21) Appl. No.: 15/116,556

(22) PCT Filed: Feb. 4, 2015
(86) PCT No.: PCT/AU2015/000055

§ 371 Date: Aug. 4, 2016

(30) Foreign Application Priority Data
Feb. 4, 2014 (AU) 2014900325

(51) Int. Cl. A61B 8/00; A61B 8/08; G01S 15/89; A61B 8/14

(52) U.S. Cl. CPC A61B 8/4461; A61B 8/145; A61B 8/4281; A61B 8/5207; A61B 8/5269; A61B 8/4494; A61B 8/4455; A61B 8/4427; G01S 15/8922

(57) ABSTRACT

An ultrasound transducer probe unit (106) is disclosed. In an embodiment, the probe unit (106) includes a body (108) portion, at least one transducer element (201) for transmitting and receiving ultrasonic signals and a lens element (1200) encapsulating a fluid medium to couple the ultrasonic signal from the at least one transducer element (201). The lens element (1200) includes at least one textured surface (1202) adapted to reduce reflections of the transmitted and/or received ultrasound signals.

This application claims priority from Australian Provisional Patent Application No 2014900325 filed on 4 Feb. 2014, the entire contents of which are herein incorporated by reference.

### FIELD

The present invention relates to medical ultrasound imaging systems. In a typical application embodiments of the present invention may be used in a hand-held real time medical ultrasound imaging system.

### BACKGROUND

Ultrasound is a non-invasive technique for generating image scans of interior body organs. There are a number of types of real time ultrasound transducers. These can be divided into electronic systems such as phased array, curvilinear array, and linear array transducers which employ fully electronic techniques for beam forming and for directing the ultrasonic beam; and mechanical systems where a transducer or a transducer array is moved mechanically to direct the beam.

Mechanical scanners are traditionally the simplest and least expensive types of real time imaging systems. These systems utilize one or more piezoelectric crystals as transducer elements which transmit the sensing ultrasound signal, and receive the echoes returned from the body being imaged.

To be effective, the ultrasound signal has significant directionality and may be described as an ultrasound beam. An electromagnetic motor is employed to move the crystal in a repetitive manner in order for the beam to cover an area to be imaged. The motor may be of any type, depending upon the movement characteristics required. Devices using stepping motors, DC motors and linear motors are known. The advantage of mechanical scanners is low cost, and relatively low power consumption. The disadvantage includes reverberation artifacts, and a limitation of the scan line density in the image due to the speed of the motor and time of flight of the ultrasound pulses.

In general, mechanical ultrasound scanners employ one of two techniques for moving the beam and generating an image.

A first technique involves a rotating wheel transducer where one or more transducer elements are rotated through 360° such that a beam emitted from the crystal would sweep out a circle. A sector of that circle constitutes the area to be imaged. The ultrasound signal is only transmitted and received while that sector is being swept out, or 25% of the time for a 90° image, limiting the number of scan lines in an image as the motor speed will be relatively high.

A second type of mechanical scan transducer involves an oscillating transducer where a single transducer element is moved back and forth such that the ultrasound beam sweeps out the region of interest. The advantage is the system can transmit and receive scan lines 100% of the time. The disadvantage is the angular velocity of the acoustic crystal is not linear, resulting in an uneven maximum scan line density limited by the maximum angular velocity.

Handheld ultrasound scanners with mechanical scan transducers traditionally use a single transducer element to transmit and receive ultrasound.

It would be desirable to provide a handheld ultrasound scanner which reduces reverberation artifacts so as to provide improved image quality. It would also be desirable to provide a hand handheld ultrasound scanner which provide an increased number of scan lines in an image or at least a more uniform scan line density.

The reference to any prior art in this specification is not, and should not be taken as, an acknowledgement of any form of suggestion that such prior art forms part of the common general knowledge.

### SUMMARY OF THE INVENTION

According to a first aspect of the present invention there is provided an ultrasound transducer probe unit including:
a body portion;
at least one transducer element for transmitting and receiving ultrasonic signals; and
a lens element encapsulating a fluid medium to couple the ultrasonic signal from the at least one transducer element with a body to be imaged;
wherein the lens element includes at least one textured surface adapted to reduce reflections of the transmitted and/or received ultrasound signals.

In an embodiment, the textured surface includes dimples having a dimple width of less than ¼ the wavelength of an ultrasound pulse of the ultrasonic signals. Preferably, the dimples have a depth which is greater than ¼ the wavelength of the ultrasound pulses of the ultrasonic signals. It is possible that other types of textured surfaces may be suitable.

The fluid medium may include an oil based fluid medium.

An advantage of an embodiment of the present invention which includes a lens having a dimpled surface is that it may act like an anechoic surface, effectively scattering ultrasound pulse reflections, but which permits ultrasound energy to be transmitted through the lens.

In one embodiment, the lens includes two textured surfaces. For example, one embodiment of the present invention includes a lens having a front textured surface and a rear textured surface.

In an embodiment, the ultrasound transducer probe unit includes a motor for moving the at least one transducer element with respect to the body portion in a repetitive motion in order to isonify an area. It is possible that the motor may be coupled to, or include, a mechanical gear of which provides a desired maximum angular velocity of the at least one transducer element.

In preference, the transducer element movement is driven by a rotary motor.

In preference, the motor interoperates with an electrical brake to enable the transducer position to be returned and stopped at a known reference angle.

Another aspect of the present invention provides a drive mechanism for a mechanically scanned ultrasound transducer, the drive mechanism including:
a motor having a drive shaft;
a pivotally mounted transducer arrangement having a pivot axis; and
a coupling for translating rotational movement of the drive shaft into reciprocating motion of the transducer arrangement over an angular extent about the pivot axis;
wherein the coupling includes a pin member which is rotated by the drive shaft through a circular path of motion, and a substantially elliptical slot disposed on the transducer arrangement for receiving the pin member, and wherein the pin member cooperates with the slot over the circular path of motion to generate the reciprocating motion.

It is preferred that the elliptical slot has a perimeter which is selected to provide a desired maximum angular velocity of the transducer arrangement so as to improve the uniformity of a scan line density associated with an image acquired by the transducer arrangement.

Yet another embodiment of the disclosure provides an ultrasound transducer probe unit including:
a body portion; and
at least two transducer elements for transmitting and receiving ultrasonic signals;
wherein the received ultrasound signals are processed to dynamically focus the signals to improve image quality.

### BRIEF DESCRIPTION OF THE DRAWINGS

An illustrative embodiment of the present invention will be discussed with reference to the accompanying drawings wherein:

FIG. 1 is a schematic diagram of an ultrasound scan system including an embodiment of the invention;

FIG. 2 is a block diagram of a probe unit suitable for use with a hand held ultrasound system of the invention;

FIG. 3 is a block diagram of a drive arrangement suitable for use with the ultrasound scan system shown in FIG. 1;

FIG. 4 is a block diagram of the functional blocks of a probe unit controller suitable for use with the ultrasound scan system shown in FIG. 1;

FIG. 5A illustrates a block diagram of a dynamic receive focus controller suitable for use with the ultrasound scan system shown in FIG. 1;

FIG. 5B is a functional block diagram of a processor slice of the processor shown in FIG. 5;

FIG. 6 illustrates a diagram of a scan line with relative delays;

FIG. 7 illustrates a diagram of the processing functions of the ultrasound scan system;

FIG. 8 is a diagram of the scan line density associated with a single slot mechanical gear mechanism;

FIG. 9 is a displacement and velocity plot of the displacement and velocity generated by an embodiment of a single slot mechanical gear mechanism for use with the present invention;

FIG. 10 is a diagram of the scan line density associated with an elliptical single slot mechanical gear mechanism;

FIG. 11 is a displacement and velocity plot of the displacement and velocity generated by an embodiment of an elliptical slot mechanical gear mechanism for use with the present invention;

FIG. 12 illustrates a diagram of the cross section of the transducer lens surface; and

FIG. 13 illustrates a diagram of the elliptical slot shape.

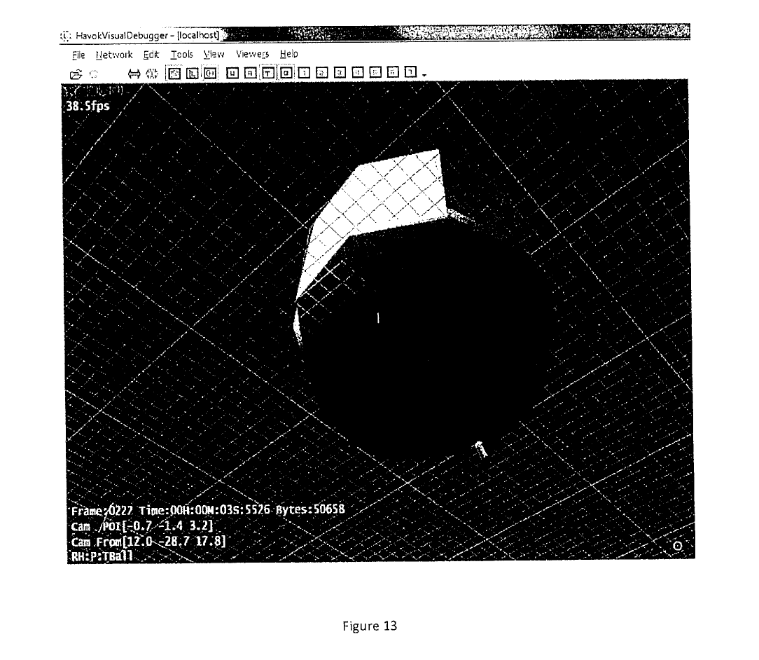

In the following description, like reference characters designate like or corresponding parts throughout the several views of the drawings.

### DESCRIPTION OF PREFERRED EMBODIMENTS

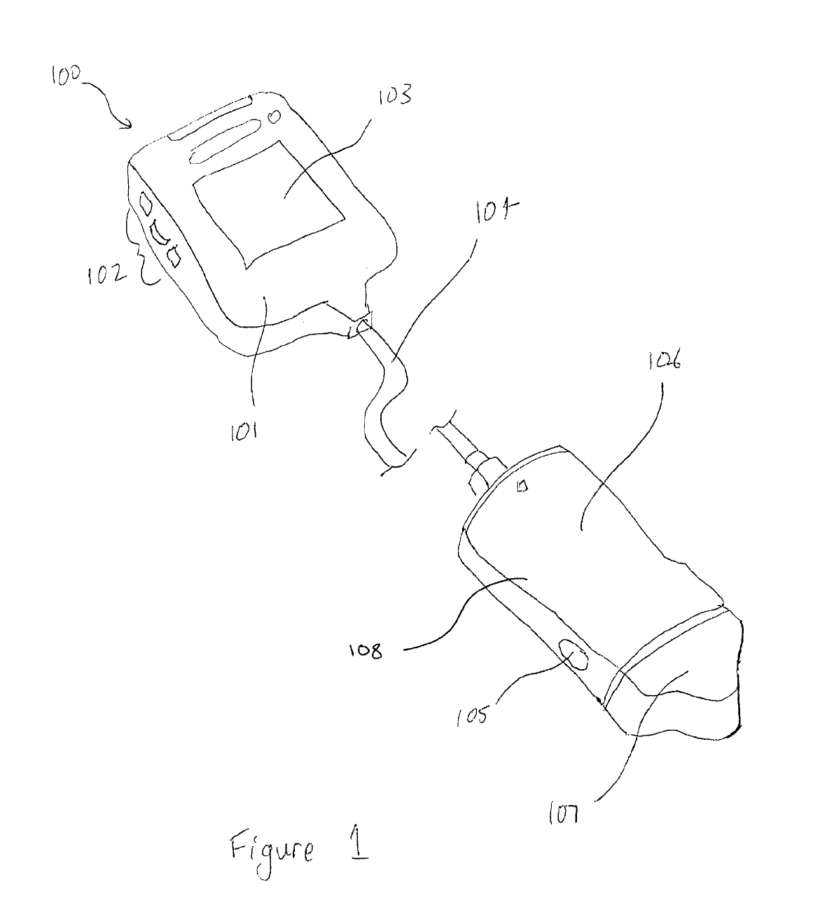

Referring now to FIG. 1, there is illustrated an ultrasonic scan system 100 according to an embodiment of the invention. As shown, the system 100 includes a hand held ultrasonic probe unit 106 having a body 108, a display and processing unit (DPU) 101 including a display screen 103 and a cable 104 connecting the probe unit 106 to the DPU 101. User controls 102 are also provided.

The display screen 103 may include, for example, a touch screen allowing a user to control the functionality of the display screen 103 and the probe unit 106. In the illustrated embodiment, user controls 106 are provided on the display and processing unit 101, in the form of push buttons and a scrollwheel. It is not essential that user controls 102, 105 be provided.

The probe unit 106 includes an ultrasonic transducer head 107 containing at least one transducer element (not shown) mechanically coupled to a motor (not shown). Each transducer element is controlled to transmit pulsed ultrasonic signals into a medium to be imaged and to receive returned echoes from the medium to be imaged. In the present case, the ultrasonic transducer head 107 includes eight transducer elements arranged in an annular array. However, it is possible that a different number of transducer elements may be used. In use, the probe unit is held against the body of a patient adjacent to the internal part of the body which is to be imaged, with the transducer head 107 in contact with the patient's skin. Electronics in the probe unit 106 stimulate the emission of an ultrasound beam from the transducer elements. This beam is reflected back to the transducer as echoes by the features to be imaged. The transducer receives these echoes which are amplified and converted to digital scanline data. The transducer is moved by an ultrasonic motor (USM) in order that the beam can cover all of a selected planar area within the patient's body. The scanline data is then displayed on the display screen 103 as an ultrasound image.

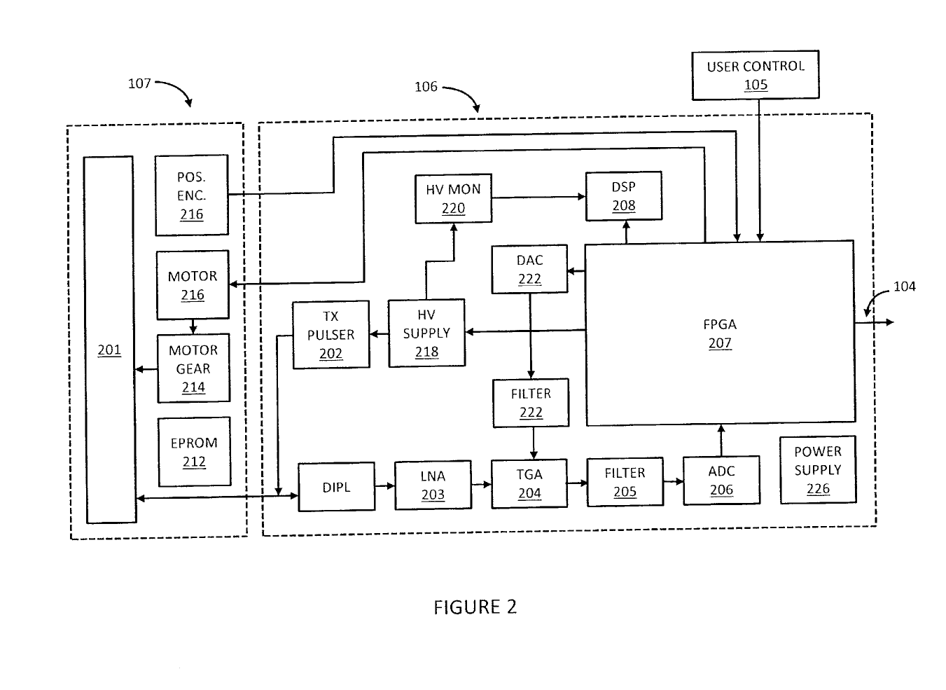

Turning now to FIG. 2, there is shown a block diagram of the probe unit 106 according to an embodiment of the disclosure. As shown, the transducer head 107 includes at least one transducer element 201, EPROM 212, motor position encoder 209, and motor gear 214, and motor 216. The operation of the motor 216 will be described in more detail later.

As shown, the probe unit 106 includes transmit pulser 202, low noise amplifiers 203, time gain amplifier 204, filters 205, 224, Analog to Digital (A/D) converter 206, digital signal processing device 208, Field Programmable Gate Array 207, HV supply 218, HV monitor 220, and Digital to Analog (DAC) converter 222.

In the present case, in operation the transmit pulser 202 generates a short electrical pulse to create an oscillation in eight transducer elements 201. Each transducer element 201 generates an ultrasonic pressure pulse which is transmitted into the medium to be imaged. The eight transducer elements 201 then receive any reflected ultrasonic pressure pulses and convert the received pressure pulse into received electrical signals.

Low noise amplifiers 203 then amplify the received electrical signals for further signal conditioning, which in the present case involves applying time gain amplification 204, and filtering the output of the time gain amplifier 204 using a bandpass or low pass filter 205 to provide an analog output signal. The analog electrical output signal is then converted to a digital output via an A/D converter 206. In the present case, digital output values of the A/D converter 206 are input to a field programmable gate array (FPGA) 207 in a low voltage serial format to reduce the number of printed circuit board traces.

The input digital values are deserialised by the FPGA 207, preferentially delayed to provide receive focussing, buffered and transferred to the digital signal processing (DSP) device 208 as raw scanline data. The entire process of receiving reflected pulses and transferring the scanline data to the digital signal processing device 208 is defined as acquiring a scan line.

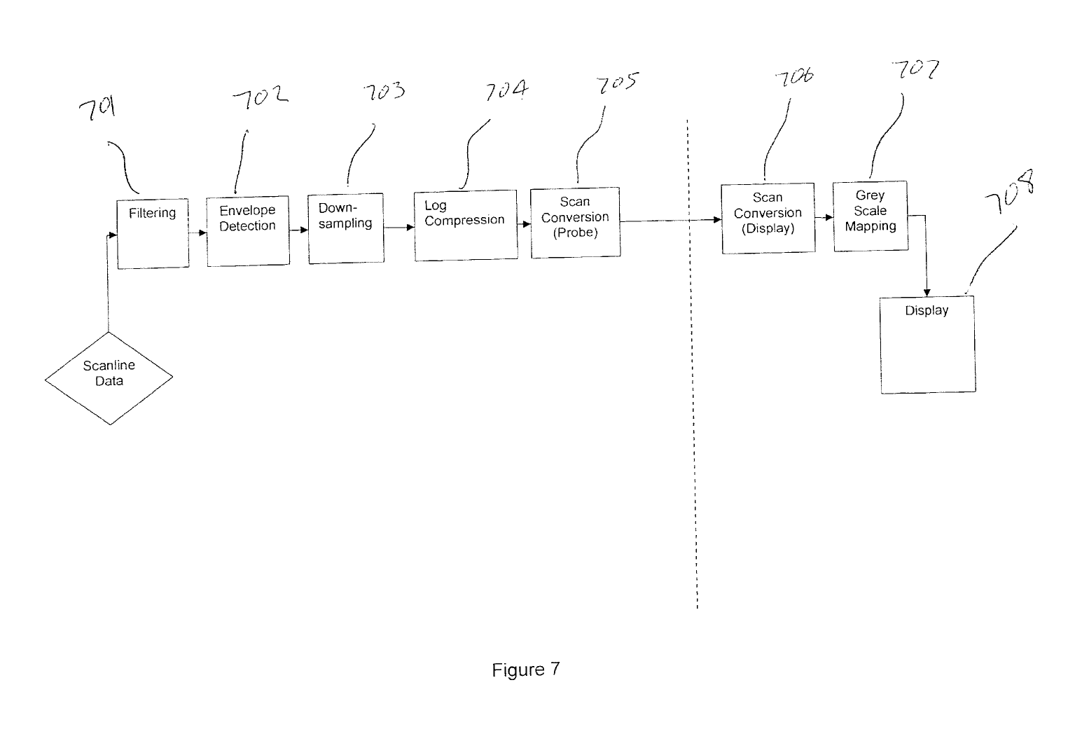

Referring now to FIG. 7, the digital signal processing device 208 processes each individually acquired scanline by applying a digital filter 701 to the scanline data, detecting the envelope of the scan line data 702, downsampling the enveloped data 703, compressing the raw input data which is preferably 12-bits into a low number of bits 704, and storing the scanline for later scan conversion by a scan converter 705/706 as will be described in more detail later.

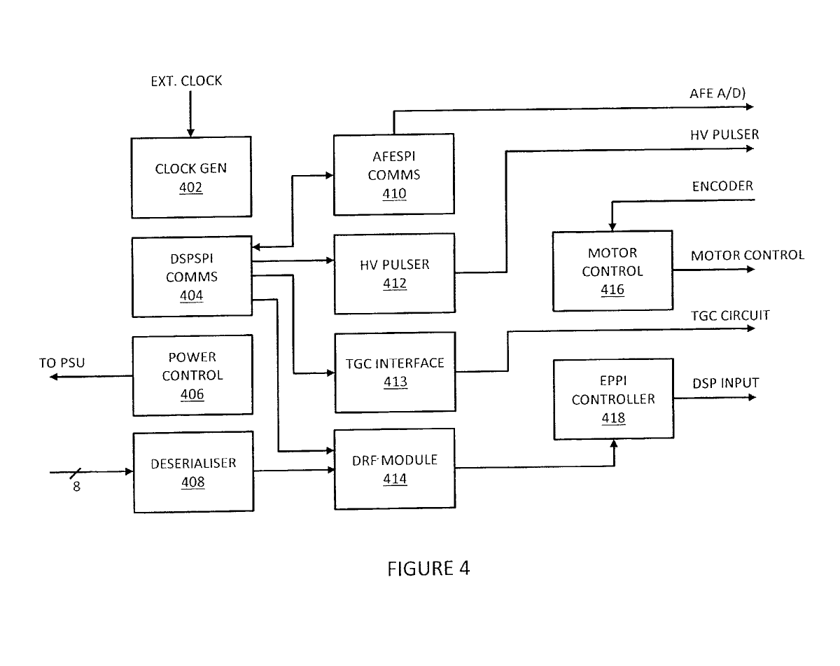

At the completion of a scan line transmit, acquisition, and processing, the FPGA 207 awaits the appropriate time to transmit the next pulse and repeat the process. The timing of the transmission of a pulse is thus controlled by the FPGA 207, as will be explained in more detail below with reference to FIG. 4.

FIG. 4 shows a block diagram of the functional modules of the FPGA 207. As shown, the functional modules include a clock generator 402, DSPSPI Comms module 404, power control module 406, deserialiser 408, AFESPI comms module 410, HV pulser interface module 412, TGC interface module 413, Dynamic Receive Focus (DRF) module 414, motor controller module 416, and EPPI controller module 418.

The DSPSPI Comms module 404 contains configuration memory setup by the DSP 208 (ref. FIG. 2) prior to performing a scan. This configuration memory may include a scanline firing table containing an encoder count for each scanline acquisition. For example, if 128 scanlines are required to generate a sector image, the scanline table contains 128 entries with the encoder position for each scanline. During scanning, the motor controller module 416 of the FPGA 207 (ref. FIG. 2) receives motor position data from the motor position encoder 209 as encoder input, converts the encoder input to a count, and compares the count to the DSPSPI Comms module 404 scanline table. When the count matches a scanline firing position, the HV pulser interface module 412 of the FPGA 207 (ref. FIG. 2) triggers the HV pulser to generate a transmit pulse to acquire a scanline.

As shown in FIG. 7, having acquired a set of scanline acquisitions covering an image area, the scanlines are packaged and transferred to the display processing unit 101 (ref. FIG. 1) for scan conversion 706, grey scale mapping 707, and display 708. Alternatively, and as is shown in FIG. 7, part of the scan conversion may be performed on the probe 705, in order to reduce the amount of data to send to the display processing unit, and therefore reduce the data transfer bandwidth required between the ultrasound probe and display processing unit.

Returning again to FIG. 4, the function of the DRF module 414 is to process eight channels of acquired data by selectively adding each channel together to provide constructive interference between channels, thereby increasing the system signal to noise ratio and improving system lateral resolution. The DRF module 414 will be described in more detail later.

In an embodiment of the present invention the transducer elements 201 are arranged in an annular array format. There may or may not be a curve on the annular array elements. However, even without a curve, due to the natural focus of any transducer element 201 there will be an effective curve. To enable each element 602 to be constructively added, the relative delay from a sample point 601 to every element must be known or calculated.

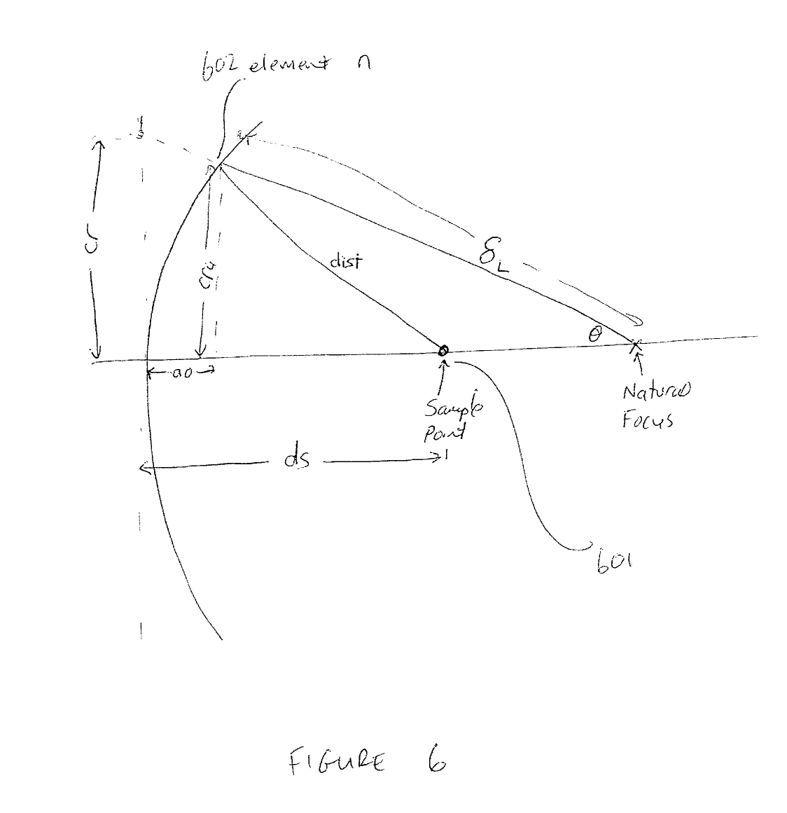

The relative delay is directly related to the relative distance. Referring to FIG. 6, the distance from a sample point to an element is referred to as dist. Dist can be calculated using a right angle triangle bounded by dist, centre radius adjusted (cra), and distance from the centre of the array (ds) less the annular offset (ao) of the element. This relationship may be expressed as:

dist=\sqrt{(ds−ao)²−cra²}

Both ao and cra can be calculated from the geometry of the annular array as follows:

$$tan θ = \frac{cr}{{δ}_{L}}$$

$$Therefore$$

$$θ = atan ( \frac{cr}{{δ}_{L}} )$$

$$And$$

$$sin θ = \frac{cra}{{δ}_{L}}$$

$$Therefore$$

$$cra = {δ}_{L} sin ( atan ( \frac{cr}{{δ}_{L}} ) )$$

$$Also$$

$$cos θ = \frac{{δ}_{L} - ao}{{δ}_{L}}$$

$$Therefore$$

$$ao = {δ}_{L} - {δ}_{L} cos ( atan ( \frac{cr}{{δ}_{L}} ) )$$

For a predefined sample frequency, a known geometry of the annular array, and a predefined interpolation oversampling factor, the relative oversampled sample delays from the centre element to all other elements can be calculated for every sample point.

The total memory required for an eight channel annular array would be the sample length (4096 samples in the preferred embodiment) times the number of element less one times the number of bytes to store each sample delay (two). This requires 56 kbytes of memory.

The amount of memory can be reduced by taking advantage of the fact that the further the sample point is from the annular array the less the sample delays change. By formatting the delay memory as eight-bits of course delay memory, two-bits of fine delay memory, and six-bits of repeat count, the required memory to store the delay parameters can be reduced by a factor of eight.

A method for dynamic receive focusing involves receiving each scan line for the eight elements, oversampling and interpolating the scan lines to enable finer resolution, delaying the scan lines by the required offset from the centre element, and then adding the scan lines together. However, as the delays change for every sample point this operation is required to be performed for every sample point.

Dynamic Receive Focussing

An embodiment of the present invention may provide a method for implementing dynamic receive focussing (DRF) which reduces resources and power consumption requirements.

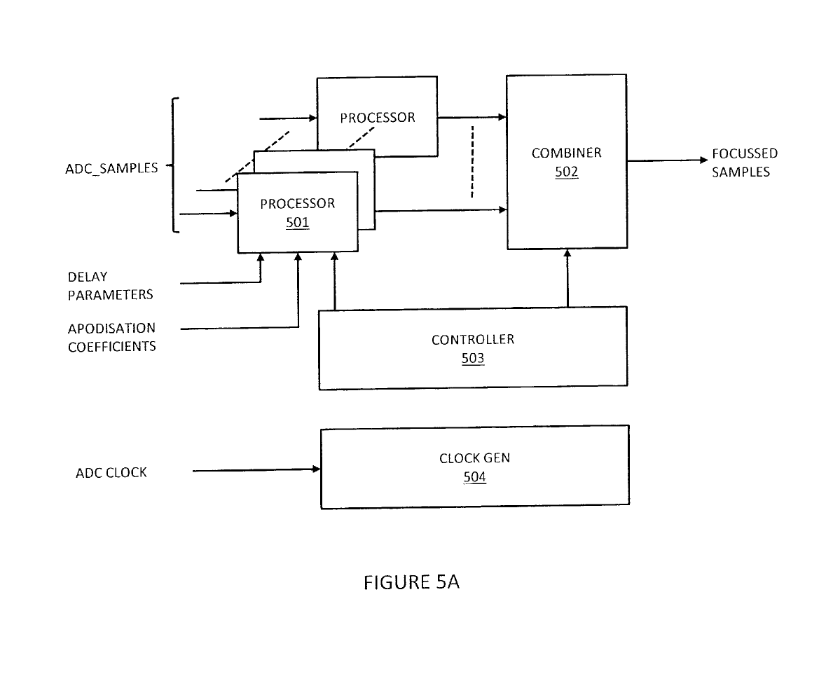

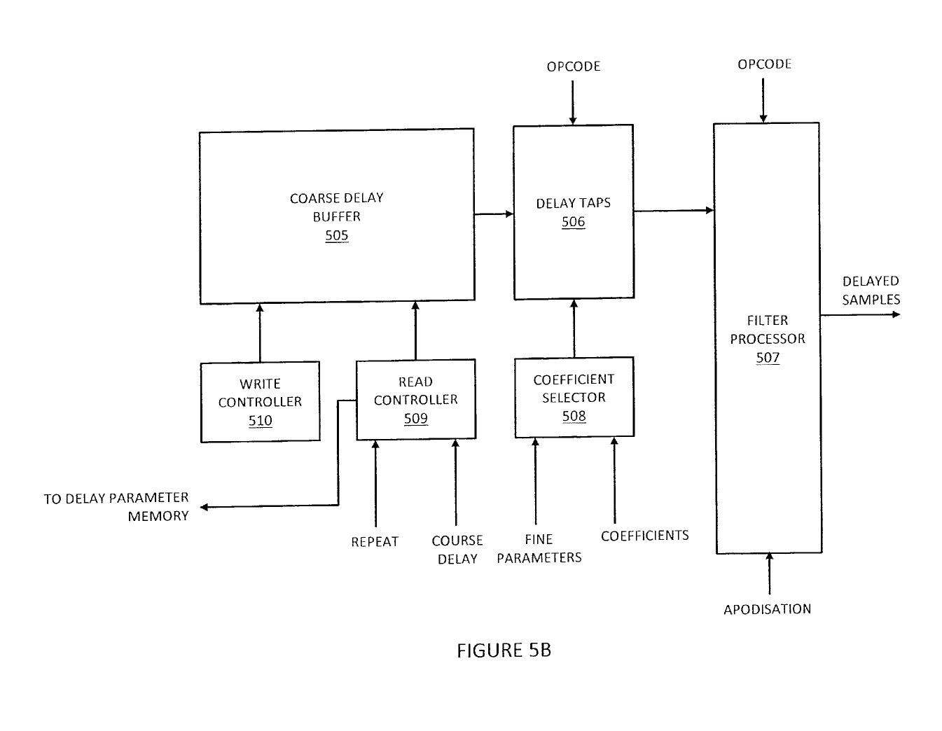

FIG. 5a shows a functional block diagram of the DRF module 414 (ref. FIG. 4) according to an embodiment of the disclosure. As shown, the DRF module 414 includes a processor slice 501, a combiner 502, a controller 503, and a clock generator 504. A processor slice 501 is required for each transducer element in the array. Hence, a preferred embodiment includes eight processor slices.

According to a method of Dynamic Receive Focussing according to an embodiment, input samples (ADC_samples) from the A/D converter 206 (ref. FIG. 2) are input to a processor slice 501 which applies a delay to the input sample. The combiner 502 adds the sample from each element together to generate a final focussed output sample. Controller 503 provides a sequencing required to perform all of the operations at the correct time, with the controller timing reference generated by the clock gen module 504 and directly related to the ADC clock (sampling clock).

A functional block diagram of a processor slice 501 is shown in detail in FIG. 5b. As shown, the processor slice 501 includes a course delay buffer 505, delay tap buffers 506, filter processor 507, coefficient selector 508, read controller 509, and write controller 510.

The write controller 510 receives input samples and writes them into the coarse delay buffer 505 having a delay buffer length. The coarse delay buffer length should preferably be of sufficient length to allow for the maximum differential sample delay between the centre transducer element and the outside transducer element. In a preferred embodiment of eight elements and a 20 mm outside transducer diameter, a buffer of 128 samples is required. The coarse delay buffer 505 is prefilled for each scanline to almost 128 samples. The read controller 509 then uses a prestored delay parameter data (prestored in the DSPSPI Comms module 402) to sequence the reading of the correct samples out of the coarse delay buffer 505. Enough samples are pre-read of out the coarse delay buffer 505 to use in the delay filters 506, 507.

The function of oversampling and interpolating each scan line requires a signal processing operating where samples are zero padded (with oversamplerate—1 zeros), and then low pass filtered. In an embodiment, a 24 tap FIR filter would be used to filter the input data. However, implementing a 24 tap FIR filter for every scanline would require prohibitive processing power.

One embodiment of the invention utilises a polyphase filter and makes use of the fact that no zero padding is required for a polyphase implementation of an upsampler, and the output from each filter bank is selected according to the required delay. Therefore, only 1/upsamplerate×total taps is required to be calculated for each sample point. Compared to a conventional FIR upsampling filter, the computation required for the preferred oversampling rate of 4 and a 24 tap filter is reduced by a factor of 16.

One implementation of a polyphase filter in the processing slice 501 is shown in FIG. 5b. For a 24 tap filter and 4× oversampling rate, a total of six samples are stored in the delay tap buffer 506. The six samples stored are defined by course delay parameters and read into the delay taps buffer 506 from the course delay buffer 505 by the read controller 509. The delay parameters are received for each sample, and the fine resolution (two bits) is used by the coefficient selector 508 to select the coefficients for the relevant polyphase filter bank. The filter processor 507 uses the buffered delay taps 506 and selected coefficients to calculate only the relevant polyphase filter bank which generates the required delay.

The read controller 509 sequences the reading of the relevant parameters from memory. The repeat count is tracked so where a delay value is repeated over multiple samples new delay parameter data is not requested. When the repeat count expires, new delay parameter data is read, and the course delay buffer 505 output is updated and the delay tap buffers 506 are refilled.

The method of dynamic receive focussing may also allow apodisation to be provided for the system. The apodisation parameters may be prestored and applied to every output sample by the filter processor 507. Any type of apodisation can be prestored and applied.

An embodiment of the invention may improve memory usage by making use of the fact delay values change more slowly the further the sample is from the transducer, and therefore delay values repeat. Some but not necessarily all embodiments of the invention may reduce power consumption and reduce processes power by using a polyphase filter to implement delays by performing the upsampling and filtering for only the relevant delay.

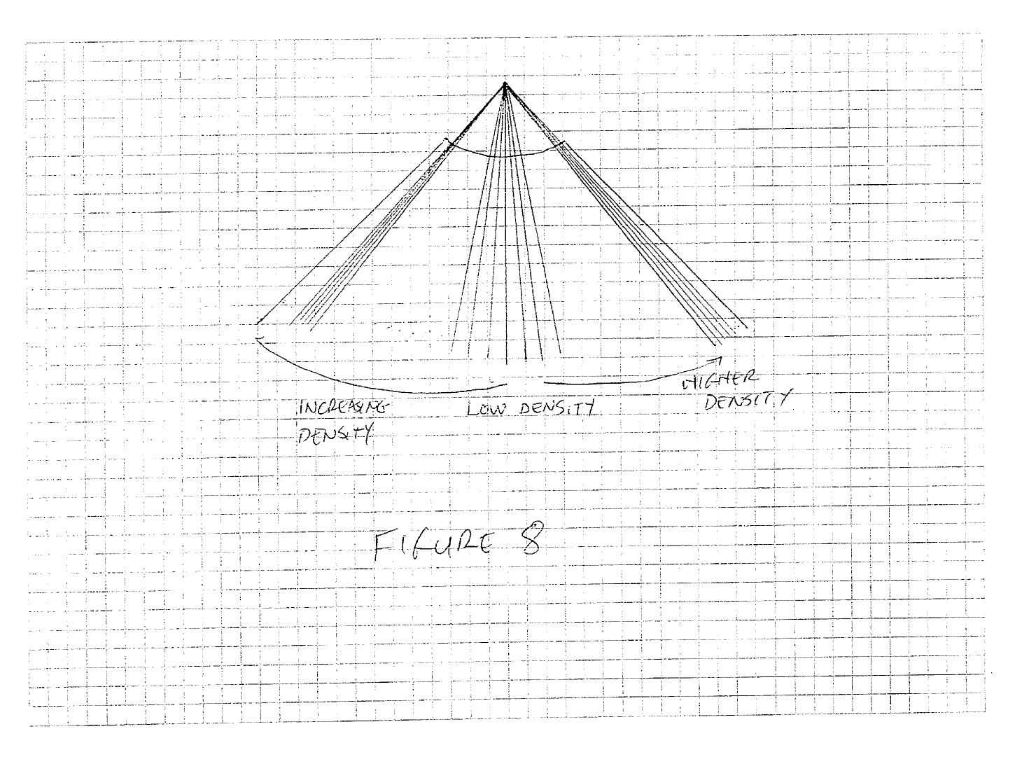

Also disclosed is a drive mechanism for a mechanically scanned ultrasound transducer. As shown in FIG. 8, the images generated by an ultrasound transducer according to embodiments of the disclosure are in a sector shape.

Drive Mechanism

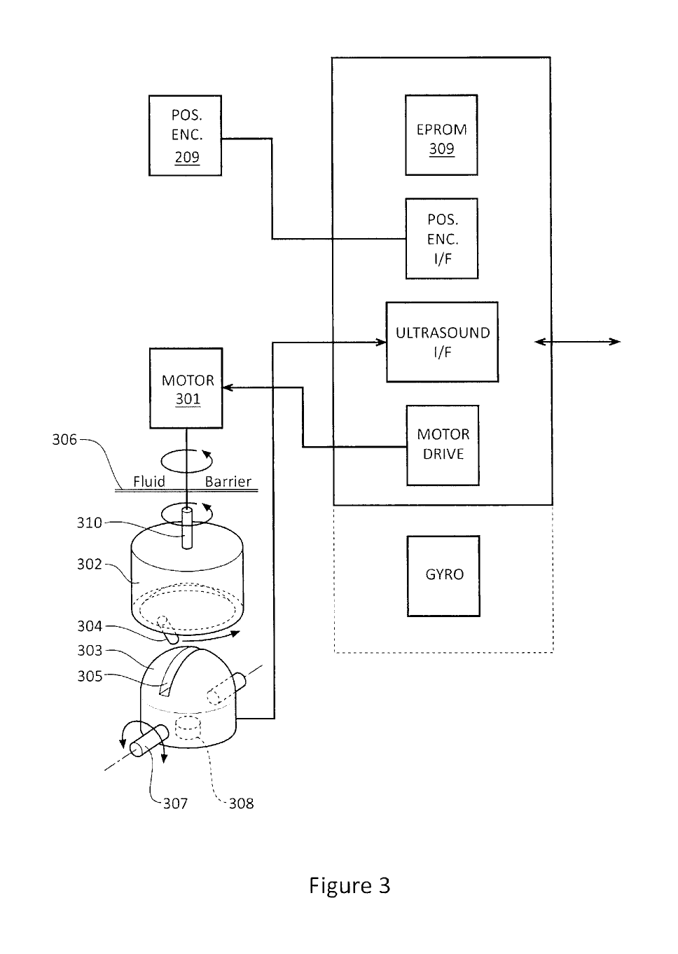

Some embodiments of the present invention may include a mechanical arrangement, in the form of a drive mechanism, which moves the transducer elements to scan a sector region so as to obtain an image of a sector. In this respect, FIG. 3 shows an example of one suitable arrangement which moves the transducer elements 308 to scan a sector region. As shown, the arrangement includes a rotary motor 301 with a shaft 310. In the present case, the shaft 310 projects through a fluid barrier 306 and is coupled to a cradle 302. The cradle 302 includes a hemispherical surface (shown dashed) and a pin 304 depending perpendicularly to the hemisphere surface at the point of connection. In the present case, rotation of the motor shaft 310 causes the cradle pin 304 to rotate about the axis of the shaft in circular motion.

In the present case, the transducer assembly 303 includes plural transducer elements 308, a hemisphere piece 303 including an elongate slot 305, and a coupling, such as shaft, which couples the transducer assembly 303 to the cradle 302 to permit the transducer assembly 303 to rotate about pivot 307 when the transducer assembly 303 is located within the cradle 302. In other words, the coupling translates rotational movement of the drive shaft into reciprocating motion of the transducer arrangement over an angular extent about a pivot axis.

The elongate slot 305 receives the cradle pin 304 such that when the motor 301 rotates, the resultant circular motion of the cradle pin 304 cooperates with the slot 305 to cause the transducer assembly 303 to rock back and forth about the pivot 307 in a reciprocating motion. In other words, the rotational movement of the cradle pin 304 is translated into a rocking or “wobbling” motion of the transducer assembly 303 over an angular extent.

For the purposes of this description the arrangement of the transducer assembly 303 within the cradle 302 will be referred to as a wobbler, and the angular displacement of the transducer assembly within the cradle 302 about the pivot 307 will be referred to as the wobbler angle. Further, the transducer assembly 303 will herein be referred to as the “wobbler ball”.

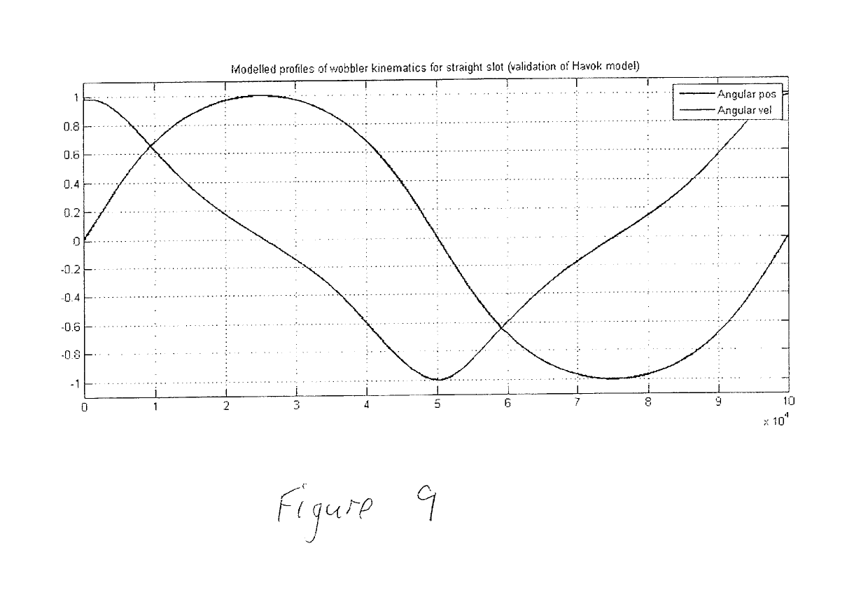

The displacement and velocity generated by the slot design are shown in FIG. 9. As shown, the velocity peaks when the transducer elements 308 and slot 305 are aligned with the motor shaft 310 (displacement shown as zero). If a scan line firing table were setup to generate transmit pulses at a fixed time period, the scan lines would be as shown in FIG. 8, where scan lines are furtherest apart at the zero angular offset, and closer together at the 45 degree angular offsets.

The mathematics for the angular velocity is as follows:

The relationship between the angle of the wobbler θ_{w} and the rotational position θ_{c} (hereinafter ‘the cradle angle’)” of the cradle pin 304:

θ_{w}=tan⁻¹(k sin(θ_{c}))

where the constant k arises from the cradle and wobbler ball geometry, specifically the wobbler ball radius r_{w} and the cradle pin 304 radial distance r_{c}:

$$k ≡ \frac{{r}_{c}}{\sqrt{{r}_{w}^{2} - {r}_{c}^{2}}}$$

Assuming a constant angular velocity of the cradle, ω_{c}, the cradle angle as a function of time is

θ_{c}=ω_{c}t

Therefore the wobbler angle as a function of time is:

θ_{w}(t)=tan⁻¹(k sin(ω_{c}t))

The wobbler angular velocity as a function of time is the derivative:

$${ω}_{w} ( t ) = \frac{d {θ}_{w}}{d t} = \frac{k {ω}_{c} cos ( {ω}_{c} t )}{1 - k {sin}^{2} ( {ω}_{c} t )}$$

This can again be differentiated to yield the angular acceleration, having the formula:

$${α}_{w} ( t ) = \frac{d {ω}_{w}}{d t} = \frac{- ( 1 - k - k {cos}^{2} ( {ω}_{c} t ) ) k {ω}_{c}^{2} sin ( {ω}_{c} t )}{{( 1 - k {sin}^{2} ( {ω}_{c} t ) )}^{2}}$$

Although the above design is effective, the limitation is a reduced number of scanlines per image due to the high velocity when the wobbler offset is zero. Hence, another embodiment of the invention may include an arrangement which reduces the maximum velocity to therefore allow an increased number of scanlines.

In one embodiment of the present invention, the slot 305 provides an ellipse slot shape having a major axis which is orthogonally to the slot direction. The co-ordinates of an ellipse slot shape are as follows:

$${r}_{c} = \frac{{r}_{1} {r}_{2}}{\sqrt{{r}_{1}^{2} {cos}^{2} {θ}_{c} - {r}_{2}^{2} {sin}^{2} {θ}_{c}}}$$

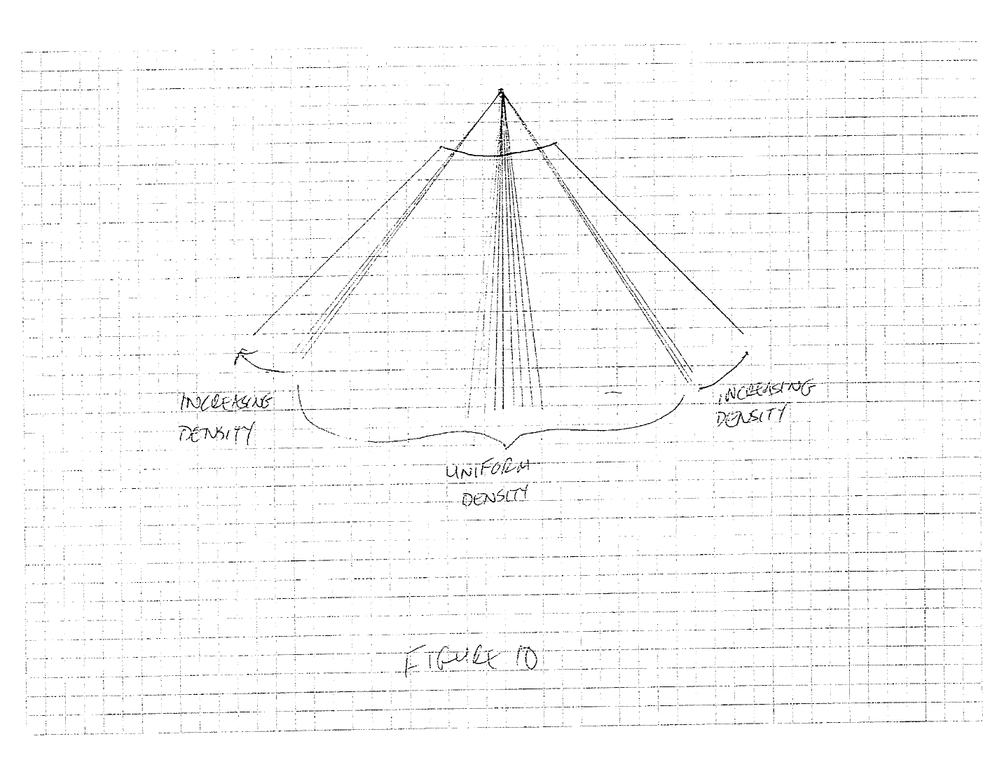

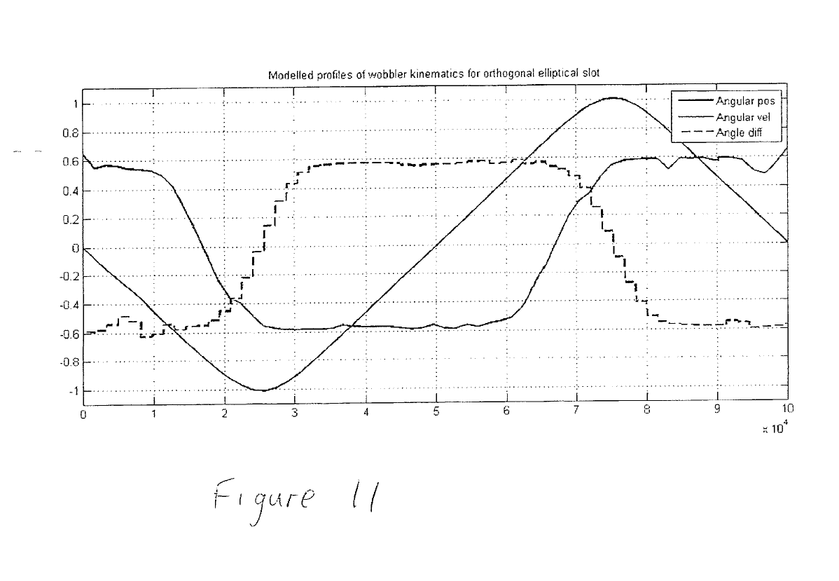

By placing the ellipse slot orthogonally to the previous slot design, the angular displacement and velocity are reduced (see FIG. 11). The maximum velocity city is reduced compared to a linear type slot design. The reduction in maximum velocity may allow more scanlines in an equivalent image. For example, with reference to FIG. 11, it can be seen that the angular velocity is constant through most of the arc and reduces at the extremities. The resulting image scan line density is shown in FIG. 10.

Textured Lens Element

Embodiments of the present invention may involve the transducer elements being immersed in a fluid medium (oil in the preferred embodiment). Unfortunately immersing transducer elements in oil may contribute to undesirable reverberation artifacts in a final image, caused by an impedance mismatch between the oil and the transducer lens. Materials may be selected for the lens element that reduce reverberation. For example, in the some embodiments TPX-MED18 may be used, where the impedance of the plastic is similar to that of the oil. However, even though similar, reflection of ultrasound pulses from the inside lens surface may still occur.

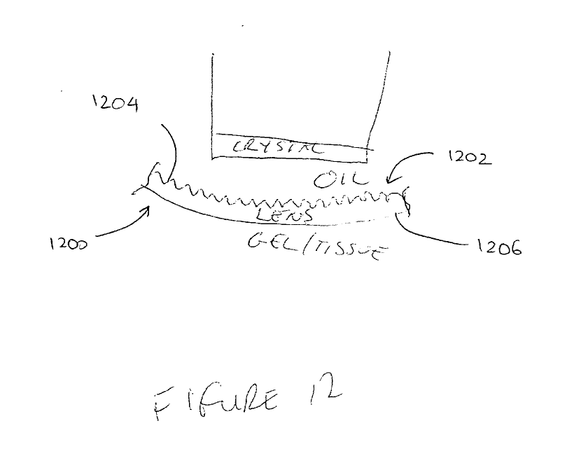

FIG. 12 shows an embodiment of the invention with the lens element 1200 constructed with a textured surface 1202, in the form of a dimpled surface, to reduce reflections. In the present case, the dimpled surface 1202 is a rear surface 1204 of the lens element 1200. However, it will be appreciated that the front surface 1206, or both the front surface 1206 and the rear surface 1204 may be a textured surface. In the present case, the textured surface 1202 includes a dimpled surface including dimples having a dimple width of less than ¼ the wavelength of the ultrasound pulse. The dimple depth is greater than ¼ the wavelength of the ultrasound pulses. The dimple design acts like an anechoic surface, effectively scattering the ultrasound pulse reflections, but as the dimples are small relative to the wavelength most ultrasound energy is transmitted through the lens.

The handheld ultrasound system is able to generate M-mode and PW Doppler images by implementing a break system on the motor. The motor design may include an electromechanical motor, with three windings pulsed in sequence to generate rotary motion. Two of the windings may be connected to a power source and switch, such that when the motor is disabled, a constant current can be provided through two of the windings generating a magnetic field that locks the motor into a fixed position. The switch is preferably a MOSFET transistor. The transducer contains an EPROM 309 with transducer information, including transducer type, transducer calibration data, and motor break calibration data. The motor break calibration data is read from the transducer before a scan, and if a scan is required to switch to a stationary scan mode, the FPGA uses the motor break calibration data to determine when to disable the motor and apply the break such that the motor breaks in a known predefined position (usually an angle offset of zero degrees).

Although preferred embodiments of the method and system of the present invention have been described in the foregoing detailed description, it will be understood that the invention is not limited to the embodiment disclosed, but is capable of numerous rearrangements, modifications and substitutions without departing from the scope of the invention as set forth and defined by the following claims.

1-13. (canceled)

14. An ultrasound transducer probe unit including:
a. a body portion;
b. a transducer element on the body portion, the transducer element being configured to transmit and receive ultrasonic signals; and
c. a lens element adjacent the transducer element, the lens element:
(1) encapsulating a fluid medium, whereby the fluid medium couples ultrasonic signals from the transducer element with a body to be imaged adjacent the lens element;
(2) including at least one textured surface configured to reduce reflections of ultrasound signals transmitted into and/or received from the body to be imaged.

15. The ultrasound transducer probe unit of claim 14 wherein the textured surface includes a dimpled surface.

16. The ultrasound transducer probe unit of claim 15 wherein the dimpled surface includes dimples having a diameter of less than ¼ the wavelength of an ultrasonic signal transmitted by the transducer element.

17. The ultrasound transducer probe unit of claim 15 wherein the dimpled surface includes dimples having a depth greater than ¼ the wavelength of an ultrasonic signal transmitted by the transducer element.

18. The ultrasound transducer probe unit of claim 14 wherein the lens element includes two spaced and distinct textured surfaces.

19. The ultrasound transducer probe unit of claim 18 wherein the two textured surfaces include a front surface and a rear surface of the lens element.

20. The ultrasound transducer probe unit of claim 14 wherein the fluid medium includes an oil based fluid medium.

21. The ultrasound transducer probe unit of claim 14 further including a drive mechanism, the drive mechanism including:
a. a motor having a drive shaft;
b. a transducer arrangement:
(1) having the transducer thereon,
(2) being pivotally mounted about a pivot axis, and
(3) having at least substantially elliptical slot disposed thereon,
c. a coupling between the drive shaft and the transducer arrangement, the coupling including a pin member which is
(1) rotated by the drive shaft through a circular path of motion, and
(2) received within the slot,
whereby rotation of the pin member over the circular path of motion cooperates with the slot to reciprocate the transducer arrangement about the pivot axis over an angular range.

22. The ultrasound transducer probe unit of claim 21 wherein the drive mechanism further includes an electrical brake configured to stop the transducer arrangement at a known reference angle.

23. The ultrasound transducer probe unit of claim 14:
a. having at least two of the transducer element, and
b. further including a processor configured to process the received ultrasound signals to dynamically focus the ultrasound signals, whereby image quality is improved.

24. A drive mechanism for an ultrasound transducer, the drive mechanism including:
a. a motor having a drive shaft;
b. a transducer arrangement:
(1) having the transducer thereon,
(2) being pivotally mounted about a pivot axis, and
(3) having at least substantially elliptical slot disposed thereon,
c. a coupling between the drive shaft and the transducer arrangement, the coupling including a pin member which is
(1) rotated by the drive shaft through a circular path of motion, and
(2) received within the slot,
whereby rotation of the pin member over the circular path of motion cooperates with the slot to reciprocate the transducer arrangement about the pivot axis over an angular range.

25. The drive mechanism of claim 24 wherein the elliptical slot has a perimeter selected to provide a desired maximum angular velocity of the transducer arrangement.

26. The drive mechanism of claim 25 wherein the desired maximum angular velocity is selected to improve the uniformity of a scan line density associated with an image acquired by the transducer arrangement.

27. The drive mechanism of claim 24 wherein the motor includes a rotary motor.

28. The drive mechanism of claim 24 further including an electrical brake for stopping the transducer arrangement at a known reference angle.

29. An ultrasound transducer probe unit including:
a. a body portion; and
b. at least two transducer elements on the body portion, the transducer elements being configured to transmit and receive ultrasonic signals, wherein the received ultrasound signals are processed to dynamically focus the ultrasound signals to improve image quality.
